(19) United States
(12) Patent Application Publication
(10) Pub. No.: US 2021/0221842 A1
(43) Pub. Date: Jul. 22, 2021

(54) PROCESS CONTROL SYSTEM FOR CONTROL AND REGULATION OF A MODULAR PLANT FOR THE PRODUCTION OF BIOPHARMACEUTICAL AND BIOLOGICAL MACROMOLECULAR PRODUCTS

(71) Applicant: Bayer Aktiengesellschaft, Leverkusen (DE)

(72) Inventors: Peter SCHWAN, Leverkusen (DE); Martin LOBEDANN, Koeln (DE); Jens BERNSHAUSEN, Koeln (DE)

(21) Appl. No.: 17/192,384

(22) Filed: Mar. 4, 2021

Related U.S. Application Data
(62) Division of application No. 15/573,148, filed on Nov. 10, 2017.

(30) Foreign Application Priority Data
May 13, 2015 (EP) 15167538.6

(51) Int. Cl. C07K 1/36; C12M 1/00; C12M 1/36; G05D 7/06

(52) U.S. Cl. CPC C07K 1/36; C12M 29/10; G05B 11/42; G05D 7/06; C12M 41/48

(57) ABSTRACT

The invention relates to a modular production plant for continuous production and/or preparation of biopharmaceutical products, a computer-implemented method for process control of the modular plant for production of biopharmaceutical and biological macromolecular products, in particular of proteins, e.g. monoclonal antibodies, vaccines, nucleic acids such as DNA, RNA and plasmids and derivatives thereof.

### CROSS-REFERENCE TO RELATED APPLICATIONS

This application is a Divisional of U.S. application Ser. No. 15/573,148, filed on Nov. 10, 2017, which is a National Stage entry of International Patent Application No. PCT/EP2016/060369, filed May 10, 2016, which claims priority to European Patent Application No. 15167538.6, filed May 13, 2015. The disclosure of the priority applications are incorporated in their entirety herein by reference.

### BACKGROUND

### Field

The invention relates to a modular production plant for continuous production and/or preparation of biopharmaceutical products, a computer-implemented method for process regulation of the modular plant for production of biopharmaceutical and biological macromolecular products, in particular of proteins, e.g. monoclonal antibodies, vaccines, nucleic acids such as DNA, RNA and plasmids and derivatives thereof. The strictly regulated production of pharmaceuticals requires major time, technical and personnel inputs for the preparation of cleaned and sterilized bioreactors and for ensuring a sterile product. In order reliably to avoid cross-contaminations during a product change in a multipurpose plant or between two product batches, apart from the cleaning, a very laborious cleaning validation is needed, which it may be necessary to repeat in the event of a process modification.

This applies both for upstream processing USP, i.e. the production of biological products in a bioreactor and also for downstream processing DSP, i.e. the purification of the fermentation products.

### Description of Related Art

The downtime of the reactors necessitated by the preparation procedures can be of the same order of magnitude as the reactor availability, particularly with short utilization periods and frequent product changes. In the USP, the biotechnological production process, e.g. the process steps of media production and fermentation, and in the DSP the solubilization, freezing, thawing, pH adjustment, product separation, e.g. by chromatography, precipitation or crystallization, buffer exchange and virus inactivation, are affected.

In order to meet the requirement for rapid and flexible recharging of the production plant while maintaining maximal cleanliness and sterility, designs for continuous production preferably with single-use technology are the subject of constantly increasing interest on the market.

WO 2012/078677 describes a method and a plant for continuous preparation of biopharmaceutical products by chromatography and integration thereof in a production plant, in particular in a single-use plant. Although WO 2012/078677 provides approaches for the continuous production of biopharmaceutical and biological products, the disclosed process is in practice not adequate. In particular, WO 2012/078677 describes the use of containers (=bags) between units connected in series. Although WO 2012/078677 discloses that the continuous process must be regulated, the authors give no information as to how this regulation can be achieved. Control is also not described in detail. The containers used are defined merely by their capacity relative to the lot size and if relevant mixing properties and are not described as buffer volumes for enabling continuous process control. Use of the container in control is thus not disclosed in WO 2012/078677 and cannot be inferred therefrom.

WO2014/137903 describes a solution for the integrated continuous production of a protein substance in a production plant, comprising columns for performing the production steps, which are connected in series. WO2014/137903 discloses that the product stream in the continuous process is ideally controlled such that as far as possible each step or each unit runs simultaneously with a similar feed rate, in order to minimize the production time. WO2014/137903 discloses the use of containers between successive units, which can accommodate the product stream for a certain time. However, these are not designed on the basis of their control properties. Use of the container volumes in control is thus not disclosed and cannot be inferred therefrom.

A method for the production of biopharmaceutical and biological products usually comprises the following production steps, which are usually connected together as follows:

A. Upstream
1. Perfusion culture
2. Cell retention system,
alternative to step 1 and 2 is a fed-batch culture.

B. Downstream
3. Cell separation
4. Buffer or medium exchange preferably with concentration
5. Bioburden reduction preferably with sterile filter
6. Capture chromatography

Usually, further steps are performed for purification of the product stream, in particular:
7. Virus inactivation
8. Neutralization
9. Optionally a further bioburden reduction (with sterile filter)

In view of the high quality standards in the production of biopharmaceuticals, further steps also usually follow:
10. Chromatographic intermediate and fine purification
11. Bioburden reduction e.g. with sterile filter
12. Viral filtration
13. Buffer exchange and preferably concentration
14. Filtration with sterile filter.

### SUMMARY

A production plant in the sense of the invention comprises units for performing at least two downstream and/or upstream steps connected together in series, in which a product stream can be conveyed. According to the invention, the units are suitable for continuous or semi-continuous implementation of a step and can be operated with a continuous product stream.

### BRIEF DESCRIPTION OF THE DRAWINGS

FIG. 1 shows a schematic representation of an embodiment of the general structure of a unit, its RIO/STU and PTU, and their connection with the PCS.

FIG. 2 shows the detailed structure of a unit and its components, and their connections to the PCS as centralized control system.

FIG. 3 shows possible arrangements of master and/or slave units in the production plant.

FIGS. 4A-C schematically illustrate the structure of slave units (FIG. 4A, FIG. 4B) and a slave unit which can be temporarily operated as a master unit (FIG. 4C).

FIG. 5 shows a schematic representation of a production plant with only one master unit (step B, nB=1).

FIG. 6 shows a schematic representation of part of a further production plant comprising two master units (step F, nF=1 and step J, nJ=1).

FIG. 7 shows an example of a possible continuous process from the fermentation up to the final filtration.

FIG. 8 shows a possible continuous process in which the downstream process is not directly coupled with the fermentation, wherein the capture chromatography and the virus inactivation (VI) are two master units.

### DETAILED DESCRIPTION OF A PREFERRED EMBODIMENT

A continuous method in the sense of the application is any process, for the implementation of at least two process steps in series, in which the output stream of an upstream step is conveyed into a downstream step. The downstream step begins the processing of the product stream before the upstream step is completed. Usually in a continuous method, a part of the product stream is always conveyed in the production plant and is described as a continuous product stream. Accordingly, a continuous conveying or transfer of a product stream from an upstream unit into a downstream unit means that the downstream unit is already operating before the upstream unit is taken out of operation, i.e. that two consecutively connected units simultaneously process the product stream that flows through them. Usually, with a constant and continuous output stream of one unit, there results a constant and continuous output stream of the following unit.

If a unit operation necessitates the changing of a component for implementation of the step (also referred to as PTU), then in the sense of the invention the unit can only be operated semi-continuously. In order to enable the continuous operation of the whole process several PTU can be operated in parallel or alternating in the relevant unit, so that a quasi-continuous stream is ensured. Alternatively, the production plant should enable the partial interruption of the product stream during the changing of the unit concerned.

A hybrid method in the sense of the application is a mixture of batch and continuously operated steps, for example all steps as continuously operated steps except for the diafiltration, which is operated in batch mode.

The different units of such a production plant typically require different flow rates. In this application, a unit which predominantly determines a flow rate is described as a master unit; a master unit comprises at least one device for conveying the product stream, usually a pump or a valve, preferably a pump. The production plant can also comprise several master units.

A continuous method for production of biological products necessitates a concept for conveying the product stream from one unit to a subsequent one. The challenge here is the matching of the input and output streams of the up- and downstream unit to one another, when the flow rates do not match one another exactly, e.g. in principle fluctuate, vary in the course of the continuous operation or are simply different. In the prior art, these variations are cushioned by a container for accommodating the product stream at the start of a unit.

Typically, a production plant includes automated regulation and control of the units through a control system, especially a process control system (PCS). Typically, the control system is connected to a control and observation station as an interface via which the user can control and observe the process.

Within the automation logic of the production plant, the control system usually comprises at least one controller, typically selected from a group comprising hysteresis, PID (proportional-integral-differential) and fuzzy controllers. The different control algorithms are configured in the process control system according to the controller type:
i. Two- or multipoint control optionally with hysteresis
ii. Control by means of a set point assignment via a polygonal chain
iii. Fuzzy control
iv. PID control—statement of proportional, integral and differential component by default setting of amplification, hold time and hold-back time.

In the simplest form of automation of the units, all pump motors of the production plant are adapted to one another and controlled by manual set point specification.

In order to operate several units coordinated with one another, an adaptation of the flow rates of the units is necessary, since two pumps at the same revolution rate never pump with exactly the same flow rate. Over time, the difference in flow rate results in the fill level in the containers increasing or decreasing.

The problem therefore consists in providing a solution for the process control of a plant for the continuous production of biopharmaceutical and biological macromolecular products, which enables the utilization of different flow rates, if necessary a time-limited (partial) interruption of the product stream, without having direct effects on the continuous operation of the adjacent units.

Matching of the flow rates is effected according to the invention via the control of a characteristic state variable, the buffer volume of the production plant. The solution according to the invention is based on the measurement and control of state variables, such as for example fill level and pressure. According to the invention, the state variable buffer volume, preferably every buffer volume, is monitored by a sensor. On the basis of the sensor data, a control algorithm influences the state variable buffer volume in a closed action sequence by means of a suitable actuator.

Hysteresis control, fuzzy control or PID control, particularly preferably PID control are preferable for the control of the state variable buffer volume. Fuzzy control can for example be defined by a polygonal chain.

According to the invention, the buffer volume in a unit can be generated by use of an expandable hose or a container.

One task of the control system in the present invention is the adjustment of the flow rates such that a continuous mode of operation of the whole process is ensured and effects of malfunctions within individual units are minimized beyond the unit concerned. Propagation of flow rate fluctuations beyond a unit can thus be minimized by the implementation of suitable control algorithms. A further task of the control system consists in preventing the buffer volumes from overflowing or running empty by pausing of one or more units, e.g. for maintenance purposes.

In the sense of the application, control means the measurement of the variable to be influenced (control variable) and continuous comparison with the desired value (target value). Depending on the deviation, a controller calculates a correcting variable which acts on this control variable such that it minimizes the deviation and the control variable adopts a desired time behaviour. This corresponds to a closed action sequence.

In the comparison, regulation means the procedure in a system during which one or more input variables influence the output variables on the basis of the rules specific to the system. Characteristic of regulation is the open action path or a closed action path, in which the output variables influenced by the input variables do not act continuously, nor on themselves again via the same input variables (http://public.beuth-hochschule.de/˜fraass/MRTII-Umdrucke.pdf). This corresponds to an open action sequence.

Control and regulation of the production plant are also summarized with the term process control of the production plant by the control system.

In the sense of the application, target control of the buffer volume means that the actuator conveys the product stream into the buffer volume.

In the sense of the application, source control of the buffer volume means that the actuator conveys the product stream out of the buffer volume.

According to the invention, all components for implementing the overall process are subdivided into units. Preferably, the individual process technology steps of the whole process are designated as units. Through the assignment of the components to units, modularity of the production plant can be created. It is possible to exchange or add individual process steps, or to change their order. During this, according to the invention, with the exception of emergency shutdowns, the regulation/control, i.e. process control, of a unit accesses only internal components of a unit.

According to the invention, a device or parts of a device for implementation of a process technology step is described as a unit. In the sense of the application, a unit has one or more of the following components:
PTU, the process technology unit, comprises the components for implementation of the step (also PT component), typically hoses, filters, chromatography columns, containers, etc., which are not connected to the control system.
STU, the service technology unit, comprises all sensors and actuators of the unit (also called ST components). These are connected to the control system via a RIO. Actuators of the STU can for example be pump motors or valves and sensors e.g. UV measurement, pressure sensors or weighing devices, etc.
A component for data acquisition and processing, in the simplest case a remote I/O, or else a local intelligence, e.g. programmable logic control (PLC) or PC-based system with I/O level. The basic automation of the unit is implemented on the local control. Both system variants are referred to below as RIO.

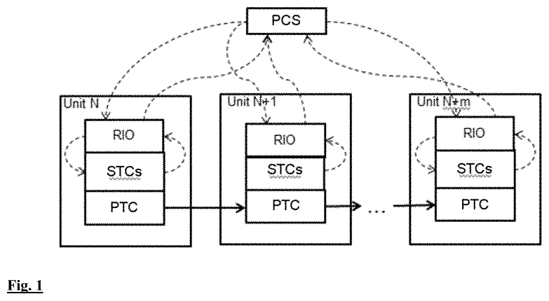

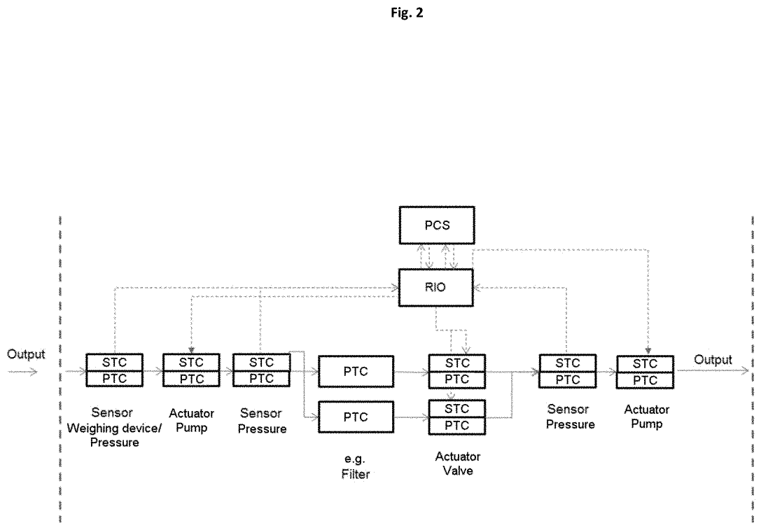

FIG. 1 shows a schematic representation of a particular embodiment of the general structure of a unit, its RIO/STU and PTU and their connection with the PCS (controllers not individually shown) without being limited thereto.

The state variable of a PTU is determined by one or more sensors of the relevant STU, such as for example the fill level of a container with a weighing device or the pressure in a filter by a pressure sensor. The STU sensor passes the corresponding signal to the RIO, which transfers this to the control system. Preferably, the signals of the STU are bundled via the RIO and transmitted to the process control system, where the corresponding correcting values are calculated.

The control system processes the signals, and calculates corresponding regulating signals, which are passed on to the connected STU actuators (e.g. motor of a pump) via the RIO. The corresponding STU actuators now act on the PTU components, which in turn react upon the STU sensors. In summary, in their interaction STU sensors, controllers and STU actuators constitute a closed action sequence for the control of the physical state variable. In the preferred embodiment, sensors of an STU serve merely for the determination of all state variables of the PTU of the same unit and result only in the regulation/control of the actuators of the same STU.

FIG. 2 describes by way of example the detailed structure of a unit and its components, and their connections to the PCS as centralized control system (controller not shown), without being limited thereto. From the previous unit, an output flows as input into the buffer volume (PTU component) of the unit. The state of the PTU component is acquired by an STU sensor, whose signals are passed on through the RIO to the PCS. The PCS sends a signal to the RIO, which passes a control signal to the motor (STU actuator) of the pump (PTU component). The product stream is passed further via hoses (PTU components) into the pressure sensor (STU sensor). The pressure signal is received in the RIO and passed to the PCS.

If the PTU is for example a filter, the product stream is passed through a first filter. If the PCS identifies that a defined pressure level before the filter has been exceeded, control signals are sent via the RIO to valves (STU actuator), which typically allow an automatic change of the filter. If the PTU is for example a chromatography column (PTU component), a change of columns would take place after a defined input volume onto the column. In this case, as the STU, a flow sensor can be used, the data from which can be integrated against time to give the input volume. Alternatively, in order to regulate the loading of product molecules onto the column, a sensor for concentration determination can be used, such as for example UV, IR . . . The integration of flow signal*concentration signal then yields the loading which if excessive would similarly lead to the change of chromatography columns.

In this preferred embodiment, the sensors, controllers and actuators acting together on the control variable, in particular, the buffer volume, are assigned to the same unit. In summary, the information flow for conveying the product stream thus usually goes along the chain STC_{N} sensor→RIO_{N}→PCS→RIO_{N}→STC_{N} actuator. The product stream passes along the chain PTC_{N}→PTC_{N+1}→PTC_{N+2} etc.

Alternatively, the sensors and/or actuators (STU actuators) for control of the buffer volume can be assigned to an adjacent (up- or downstream) unit. In this case, the information flow for conveying the product stream for example goes along the chain STC_{N} sensor→RIO_{N}→PCS→RIO_{N+1}→STU_{N+1} actuator; the product stream likewise passes along the chain PTC_{N}→PTC_{N+1}.

According to the invention, the production plant comprises several units, which are subdivided into master units and slave units.

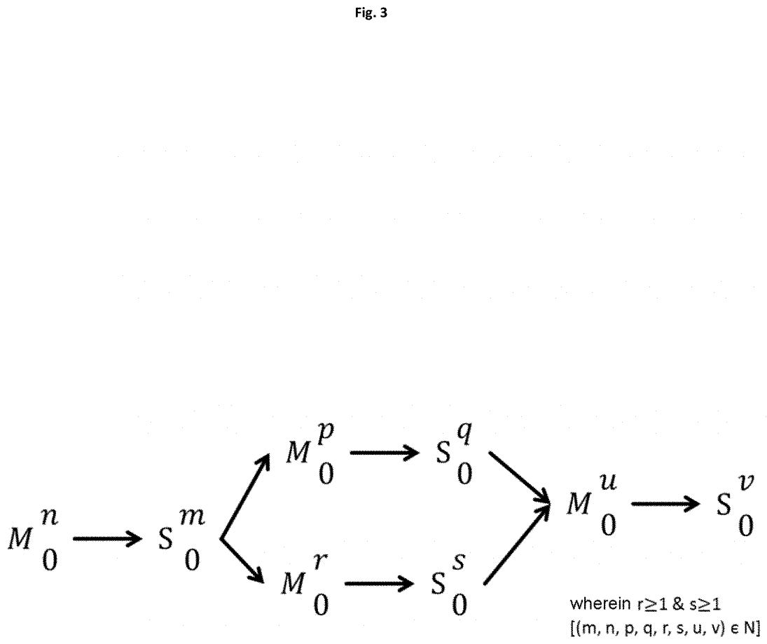

FIG. 3 shows in a general manner the possible arrangements of master and/or slave units in the production plant according to the invention.

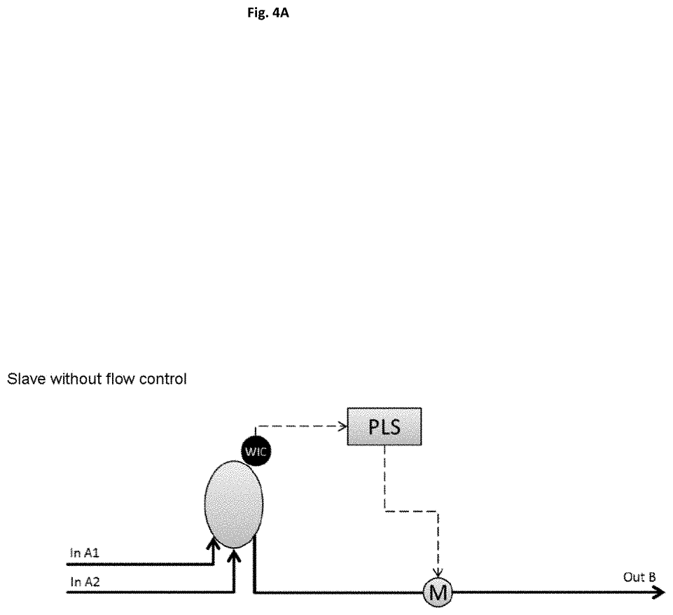

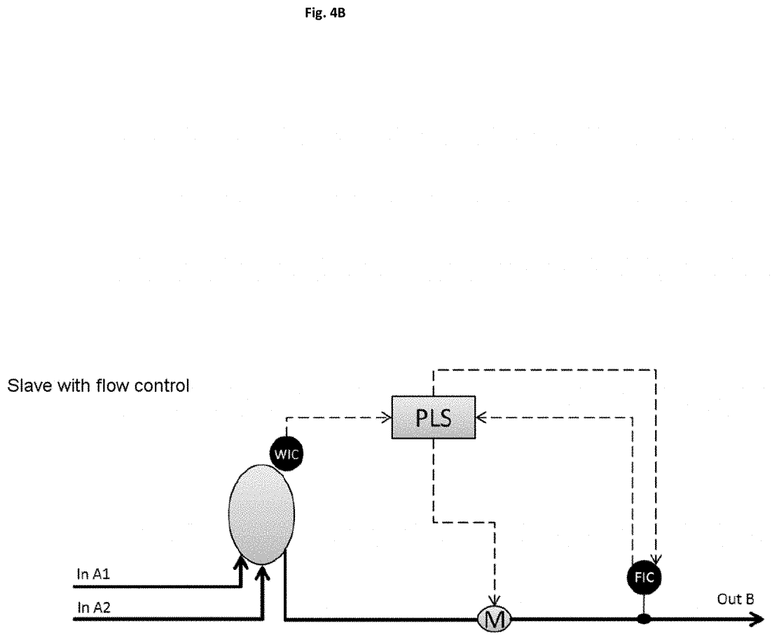

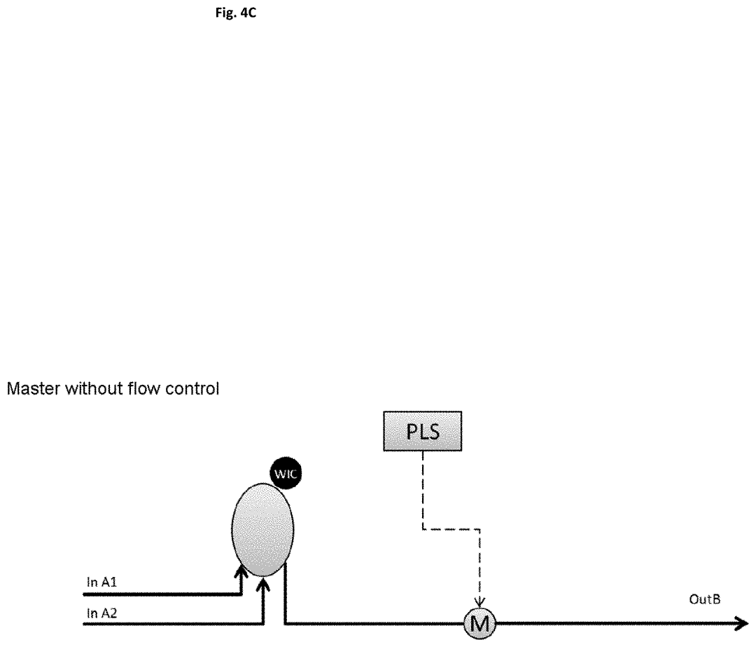

FIGS. 4A, 4B and 4C schematically illustrate the structure of slave units (4A, 4B) and of a slave unit which can temporarily be operated as a master unit (4C).

According to the invention, master unit and slave unit are defined as follows depending on their regulating or control behaviour:
The target value of the flow rate of a master unit is not obtained via the control of the state variable buffer volume. Usually it is pre-set by the control system. A master unit does not have to adapt itself to another unit with regard to its flow rate. According to the invention, a master unit comprises one or more actuators and a pipe for conveying the product stream and a RIO. Sensors e.g. for measurement and control of the flow rate are optional but preferable. When sensors e.g. for measurement and control of the flow rate of the master unit are used, the master unit is usually connected to at least one controller. This controller can preferably be part of the control system, i.e. in a centralized control, or alternatively part of a local programmable logic control (PLC) in a decentralized control. Typically, a master unit is a chromatography unit, a virus inactivation unit and/or a filtration unit.
The target value of the flow rate of a slave unit is obtained via the control of the state variable buffer volume in the same unit or in an adjacent unit along the product stream. In other words, a slave unit must adapt itself to another unit as regards its flow rate. For influencing its buffer volume, a slave unit has a closed action sequence, which is achieved by means of an STU sensor for monitoring the buffer volume (shown as WIC), a controller and an STU actuator for influencing the buffer volume (M)—all mentioned together as components for influencing the buffer volume (FIG. 4A). For controlling the state variable buffer volume, the STU sensor for monitoring the buffer volume (WIC) can be combined with a sensor for flow control (FIC) as shown in FIG. 4B.

The target value of the flow rate of a slave unit can under some circumstances, usually temporarily (e.g. in case of failure/pausing of the upstream master unit), be controlled as in the case of a master unit (FIG. 4C).

In the sense of the application, monitoring or influencing the buffer volume means monitoring or influencing the state variable buffer volume.

In the sense of the invention, the product stream which emerges from the buffer volume of each slave unit (output stream B), is typically controlled in such a manner that in spite of fluctuations of one or more input streams (input stream A1, A2), the time-averaged state variable buffer volume remains constant. The output stream B does not have to be always exactly the sum of the input streams A1 and A2.

Typically, all STU components for influencing the buffer volume are assigned to the same unit. In other words, in the preferred embodiment a slave unit comprises at least one buffer volume, at least one sensor (STU sensor) for monitoring the buffer volume and one or more actuators (STU actuators) for influencing the buffer volume. The sensors for monitoring and actuators for influencing the buffer volume are connected to at least one controller. At least one of these controllers controls the state variable buffer volume. This controller can be part of the control system (centralized control) or part of a PLC (decentralized control).

Alternatively, however, the buffer volumes, sensors, sensors for monitoring and/or actuators for influencing the buffer volume can be assigned to an adjacent (up- or downstream) unit. For example, a master unit can comprise at least one buffer volume for controlling the following unit and at least one sensor (STU sensor) for monitoring the buffer volume; the corresponding actuator for influencing the buffer volume is then assigned to the following slave unit. Such an assignment is typically effected when a chromatography unit is to be operated as a slave unit or when for reasons of space the buffer volume cannot be accommodated on the corresponding skid.

In summary, for each slave unit the production plant according to the invention comprises at least one buffer volume to accommodate the product stream and one or more sensors, controllers and actuators (STU actuators) for controlling the buffer volume either in the same unit or in an adjacent (i.e. up- or downstream along the product stream) unit.

Preferably, a source control is used within the slave units, i.e. the buffer volume is the source from which the actuator conveys the product stream. Hence in this case a master unit at the start of the plant is used.

Alternatively, a target control can be used within the slave unit, in which the buffer volume into which the actuator conveys the product stream is the target.

For reliable operation, i.e. in order to enable the shutdown of a unit during operation of the plant, the control system typically enables central monitoring of the buffer volume and enables the shutdown of a unit when needed (buffer volume too full or too empty); each master and each slave unit is connected to the control system.

The whole control system can be a combination of centralized and decentralized controls. Typical units with local control are chromatography units.

According to the invention, the buffer volume in one unit can be generated by use of an expandable hose or a container. The magnitude of the buffer volume can then be determined via the pressure or for example via the weight. The STU sensor for monitoring the buffer volume is typically a fill level sensor such as for example a pressure sensor, a weighing device, an optical sensor, etc.

Preferably each container has venting—a valve or a venting filter.

Preferably, an expandable hose is used. As the expandable hose, for example a silicone hose of the SaniPure® type was used in a test plant. As expandable hoses, Pharmed®-BPT (silicone hose), C-Flex-374® (thermoplastic hose), or SaniPure® from Saint-Gobain Performance Plastics are mentioned, without being limited thereto. Typically, a pressure sensor is used for monitoring the expansion of the hose, and thus the buffer volume. Overflow or empty running of the buffer volume is avoided in that in the control system an allowed pressure range for the buffer volume is defined, so that if the upper pressure limit is exceeded, the actuator for conveying the product stream into the buffer volume is switched off. If the lower limit is gone below, the actuator for conveying the product stream out of the buffer volume is switched off. An expandable hose is for example preferably used as buffer volume in a dead-end filtration which is connected downstream of another dead-end filtration. In this way, dead volumes in the plant can be reduced.

In an alternative embodiment, a container fill level sensor combination, in particular a container weighing device combination, is used for controlling the buffer volume.

Both embodiments enable flow rate compensation between two units, even in case of a pause or a brief stoppage of one of the two units.

Various combinations of buffer volumes and fill level sensors can be used in the same production plant.

Via the control system, the fill level in the buffer volume is controlled to a particular target value. In the test plant, the target fill levels of the containers were typically set such that the average residence time lay between 2 mins and 4 hrs, preferably about 20 mins. The target value in the case of pressure control lay between −0.5 bar and 2 bar, preferably −100 to 200 mbar, particularly preferably 10 to 50 mbar relative to ambient pressure.

In the control system, the direction of the information flow between the components, STU sensors, controllers and STU actuators which contribute to the control of a buffer volume is specified in accordance with the above-mentioned definitions and the units are thereby subdivided into master or slave units. This can be performed by the user via a user interface or in the configuration of the control system.

Preferably, the control system is programmed for automatic compatibility testing of the manual subdivision of the units in accordance with the above-mentioned definitions.

It is noted that for the assignment of the components for controlling the buffer volume in a unit or adjacent unit and/or for specifying the direction of the information flow between the components—STU sensors, controllers and STU actuators—for controlling a buffer volume, in each case only the components of each closed action sequence are taken into account. The assignment of STU components along the product stream to a unit are part of the modular structure of the production plant. The individual consideration of closed action sequences for controlling the buffer volumes in conjunction with the continuous product stream and its flow rates enables the modular structure of the regulation/control of the production plant in units according to the invention.

Hence a first subject of the application is a production plant for continuous production and/or preparation of biopharmaceutical products with at least two units connected together in series for implementation of at least two downstream and/or upstream steps, wherein the production plant comprises:
at least one slave unit and at least one master unit,
wherein each slave unit is connected to at least one buffer volume either in the same unit or in an adjacent unit along the product stream and has one or more sensors for monitoring the buffer volume and one or more actuators for influencing the buffer volume and wherein the state variable of each buffer volume is controlled by means of the sensor and the actuator connected to at least one controller in a closed action sequence,
wherein a master unit comprises at least one device for conveying the product stream and is characterized in that its flow rate is not controlled via the control of the state variable buffer volume,
and wherein, if the master unit is adjacent to one or more slave units, it is connected to the buffer volume of each slave unit, and
wherein in the case of several master units at least one auxiliary stream is present between two flow rate-determining actuators of the master units.

Preferably, one or more of the controllers are components of a control system, especially of a process control system.

In order to enable the switching off of a master unit during operation, each master unit is preferably connected to the control system.

A further subject of the application is a computer-implemented method for process control of the production plant according to the invention, wherein:
the values of the state variable buffer volume and the flow rate in the production plant are specified by the following statements:
   the order of the units along the product stream is stated,
   a target value for the flow rate is specified for each master unit,
   a target value for the state variable is specified for each buffer volume,
   for each closed action sequence, the connection of the controllers to the sensors for monitoring the buffer volume and to the actuators for influencing the buffer volume and if appropriate their connection to one another are specified,
   a parameterization of the controllers is carried out.

For the operation of the production plant, the method according to the invention comprises the following steps:
a) The target value for the flow rate of the master units is transmitted by the control system to an actuator for regulating the flow rate in the master unit, with the proviso that in the case of several master units an auxiliary stream is opened, and
b) The actual value of the state variable buffer volume is determined by the corresponding sensor for monitoring the particular buffer volume, passed on to the controller connected in the respective closed action sequence and there compared with the respective corresponding target value,
c) The respective regulating signals are calculated and transmitted to the respective actuators connected in the closed action sequence for influencing the buffer volume,
d) The actuators for influencing the buffer volume react upon the sensors for monitoring the buffer volume and
e) Steps b) to d) are repeated until the production plant is switched off or shut down.

Preferably, shutdown conditions are additionally defined by the following statement:
a maximum and/or minimum value for the state variable buffer volume is specified, preferably both,
a maximum and/or minimum value for the flow rate is specified for each master unit, preferably both.

A further subject is a computer program for implementing the above-mentioned process.

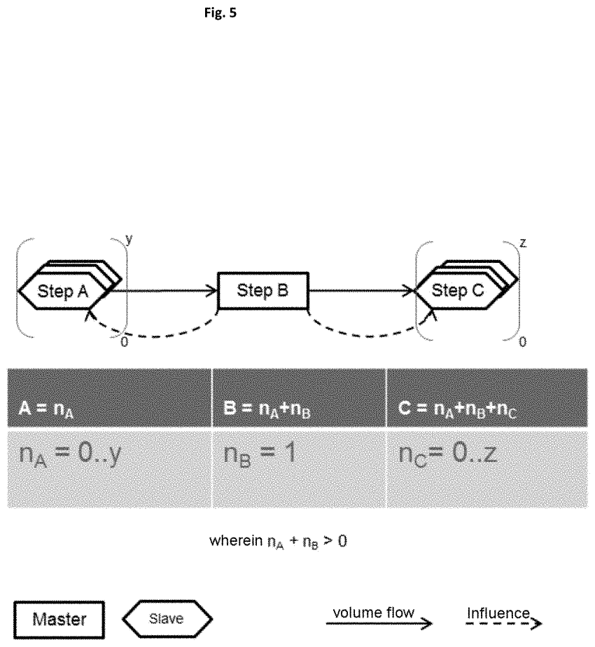

FIG. 5 shows a schematic representation of a production plant with only one master unit (Step B, n_{B}=1). The direction of the product stream and the information flow in the plant have also been correspondingly defined.

The plant can comprise n_{A}=0 to y slave units—here summarized as (Step A)_{0 . . . z}.

Likewise the plant can comprise n_{C}=0 to z slave units, here summarized as (Step C)_{0 . . . z}.

The process step number (y or z respectively) represents the last process step number in the series.

In this configuration, a slave unit (Step A or Step C respectively) can in each case stand as an individual unit at the start and/or the end of the plant.

Typically, a chromatography step is a master unit. Several chromatography steps can all act as master units, provided that an auxiliary stream is present between two master units in each case. Here, “between two master units” means behind the pump for conveying the product stream from the first master unit and the first pump for conveying the product stream in the master unit 2.

Alternatively, only one chromatography unit is operated as master unit, and the other chromatography units are each operated by means of a buffer volume as slave units and preferably controlled with a hysteresis control (centralized or local).

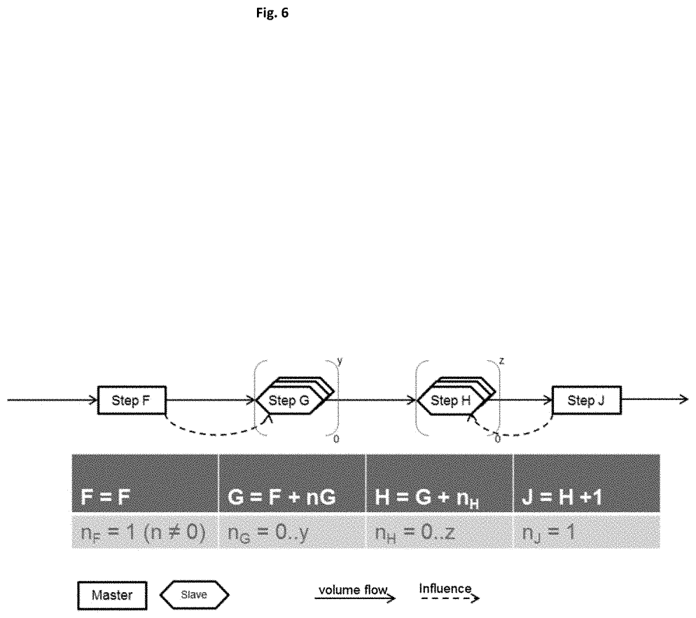

FIG. 6 shows a schematic representation of part of a further production plant comprising two master units (Step F, nF=1 and Step J, nJ=1). FIG. 6 illustrates only the part between the master units. The whole picture of the process emerges from combination with FIG. 5 for the control of the beginning and end of the process plant.

For the overall process, there is always a master flow rate (PF), which is specified externally or by a master unit, or by the first master unit in the product stream direction, if several are present.

Between two master units, at least one auxiliary stream (not shown in Fig.) must be present, since it is not possible to control two master units with exactly equal flow rate. The auxiliary stream conveys liquid into the product stream or out of the product stream (concentration). The auxiliary stream compensates the difference between the master flow rate, in FIG. 6 specified by master unit F, and the flow rate of the downstream master unit J.

Auxiliary stream in the sense of the application designates a non-product-laden (or waste product-laden) stream, which is conveyed into or out of the product stream. Auxiliary streams which are conveyed into the product stream can be controlled. Typically, one of the master units in this embodiment of the production plant comprises an STU sensor for measuring the auxiliary stream and an STU actuator for controlling and regulating the auxiliary stream, and PTU components for delivery or removal of an auxiliary stream (which are summarized as AUX-PTU components). Auxiliary streams which are removed from the product stream are usually not controlled.

If for example a continuous virus inactivation with constant input flow (master 2 with flow F2) is connected downstream of a continuous elution from a protein A chromatography (master 1 with flow F1), then an auxiliary (F3) is needed to compensate the flow rate difference, since F2>F1. F2<F1 is not useful since it leads to product loss, and F1=F2 is technically not possible without control. Flow rates F1 and F2 are not controlled, but only regulated. Flow F3 results either automatically (F3=F2−F1), or can be regulated by control of the fill level or pressure. Preferably, the flow F3 results automatically. Although the plant according to the invention has at least one master and at least one slave unit, the use of an auxiliary stream is transferable to a plant which comprises only master units.

A further typical master unit is the continuous virus inactivation according to PCT/EP2015/054698. If the plant comprises a chromatography unit and a continuous virus inactivation, an auxiliary stream can be used between the master units. In this embodiment of the chromatography unit, an auxiliary stream is always added to the product stream before the continuous virus inactivation (during operation and pausing). In order to avoid this, the chromatography unit is preferably operated as a master unit and the continuous virus inactivation as a slave unit. Here it should be noted that when the chromatography unit (master unit) is paused, the continuous virus inactivation, as a time-critical step, must be operated as master unit. This is achieved in that both an auxiliary stream for the operation of the unit for continuous virus inactivation as a master unit, and also a buffer volume for the operation of the unit for continuous virus inactivation as a slave unit, are present between the chromatography unit and the unit for continuous virus inactivation.

In a preferred embodiment of the production plant, the units for implementation of the steps in units are operated as follows:
Perfusion culture and cell retention system typically form one unit, which is typically operated as a master unit,
Concentration and dialysis positioned directly downstream can likewise together form a unit, which is operated as a slave unit. Preferably however, a filtration is performed between concentration and dialysis. In this case, they form separate slave units.
Chromatography units are typically operated as master units. However, a chromatography unit can also be operated as a slave unit, if the software for controlling the chromatography enables this, i.e. the chromatography can be run automatically at at least two different rates.
Homogenization, virus inactivation and neutralization preferably together form one unit, which is typically operated as a slave unit, but preferably when necessary temporarily as a master unit.
Filtrations—for cell separation, filtration for bioburden reduction or particle removal or virus filtration—are typically slave units.
Residence time components for reaction such as for example precipitations or also crystallizations are typically slave units, but are preferably integrated into other units. For the continuous mode of operation, a residence time component, e.g. hose, preferably coiled hose, particularly preferably a coiled flow inverter (CFI) is used.
Conditioning components for parameter setting of the product stream such as for example pH and conductivity values are typically slave units, but are preferably integrated into other units. Preferably the conditioning is effected in a conditioning loop which is attached to the buffer volume.

The units of the production plant can all be operated continuously. In this embodiment, the virus filtration is preferably performed as the last step before a bioburden reduction or as the last process step. This enables, when necessary, a fresh virus filtration of the product stream. This has the further advantage that when necessary the mode of operation of the units—virus filtration with/without bioburden reduction—can be changed from continuous to batch.

Alternatively, individual units can be operated batchwise. For example, all steps up to the virus inactivation can be operated continuously, the virus inactivation run batchwise and the further steps again run continuously, in which the buffer volume must be configured such that the continuous operation of the up/downstream units is ensured.

In the plant according to the invention, the target value of the flow rate of product-laden volume flow is usually 0.001 to 10 L/minute, preferably 0.01 to 5 L/min, particularly preferably 0.05 to 1 L/min.

The measurement of flow rates, in particular of <50 ml/min, is a challenge in a continuously operated plant. It was found that this measurement is not possible by means of commercially available, autoclavable or gamma-sterilizable disposable flowmeters. This problem can be solved in a plant with flexible pipes, in which a liquid stream is conveyed, through the use of a compensating flow rate measurement. This is solved by a combination of a compensating pump, a pressure sensor and a controller with a desired target pressure. The pressure difference between inlet and outlet of the compensating pump is kept almost constant. Preferably, this difference is zero, particularly preferably the pressure before and after the compensating pump respectively corresponds to the ambient pressure. In the event of deviations of the actual pressure from the target pressure, the revolution rate and thus the output of the compensating pump are appropriately adjusted. Finally, via the measurement of the revolution rate of the compensating pump and the conveyed volume per revolution, the flow rate can be calculated (=compensating flow rate measurement).

The magnitude of the buffer volume depends on the flow rates and the inertia of the control. If a unit requires a regular shutdown for the maintenance of a PTU component, a larger buffer volume in the form of a container is preferably used. Typical such units are chromatography.

Typically, a container has no stirrer. If mixing of the contents of a container is necessary, a stirrer can be used, but preferably the mixing is effected by a circulation system (pipe and pump).

For illustration of the process according to the invention, the configuration of various PCS for plants for upstream and downstream processing or only downstream processing of a product stream from a fermenter is shown schematically. These configurations are by way of example and do not represent any limitation of the process according to the invention.

In the figures, the production plant is subdivided into skids. According to the prior art, a skid is a three-dimensional solid structure which can serve as the platform or support of a unit. Examples of skids are shown in the figures.

### Examples

1) Fermentation->DSP I and DSP II

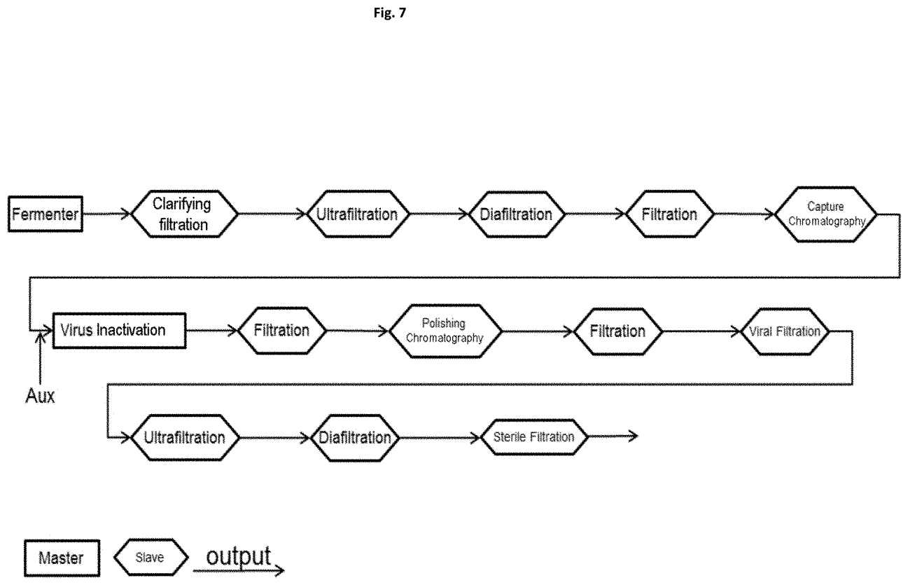

FIG. 7 shows by way of example a possible continuous process from the fermentation up to the final filtration. This production plant comprises two master units—the fermentation and the residence time-critical virus inactivation (VI). In order to be able to effect a constant time-averaged volume flow from the virus inactivation (VI), an auxiliary stream (Aux) is added after the capture chromatography, which in this example is operated as a slave. The other units are slave units.

2) Only DSP II in which according to FIG. 6, nG=nH=0

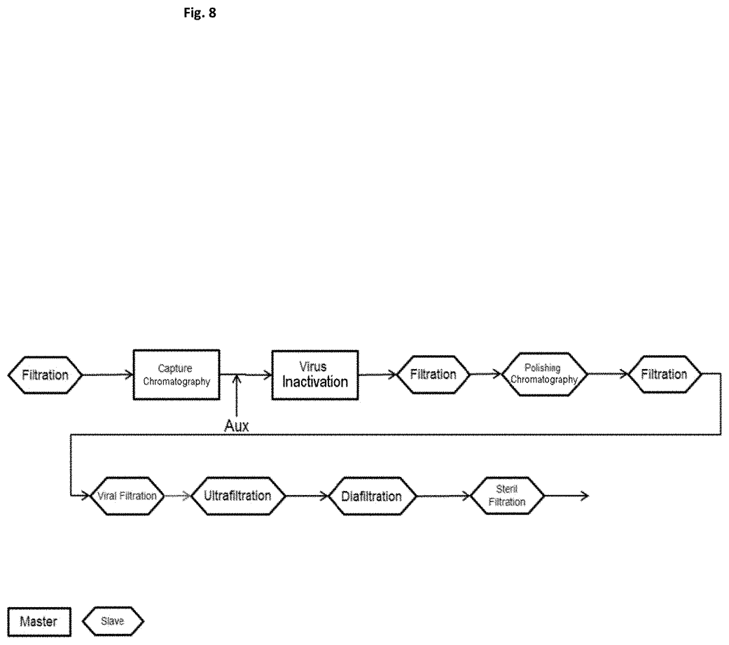

FIG. 8 shows an example in which the downstream process is not directly coupled with the fermentation, wherein the capture chromatography and the virus inactivation (VI) are two master units. In order to be able to effect a constant volume flow from the capture chromatography, an auxiliary stream (Aux) is added after this. The filtration located upstream of the capture chromatography is then a slave unit. The units located downstream are also slave units.

The studies which resulted in this application were supported in accordance with the grant agreement “Bio.NRW: MoBiDiK—Modular Bioproduction—Single-use and Continuous” in the context of the European Regional Development Fund (ERDF).

1. A production plant for continuous preparation of a biopharmaceutical product with at least two units connected together in series, wherein the production plant comprises:
at least two slave units selected from the group consisting of a concentration and dialysis unit, a filtration unit, a homogenization, virus inactivation and neutralization unit, a residence time components unit, and a conditioning component unit,
and one master unit, wherein the master unit comprises a chromatography unit,
wherein each slave unit is connected to one or more buffer volumes generated by a container and comprises one or more sensors, one or more pumps, and one or more controllers,
wherein a state variable of each of the one or more buffer volumes is controlled by the one or more sensors and the one or more pumps connected to the one or more controllers in a closed action sequence, and
wherein the master unit comprises at least one pump for conveying a product stream comprising the biopharmaceutical product and is characterized in that its flow rate is not controlled via the control of the state variable of the buffer volume.

2. The production plant according to claim 1, wherein one or more of the controllers are components of a control system, optionally a process control system.

3. The production plant according to claim 2, wherein the master unit is connected to the control system.

4. The production plant according to claim 1, wherein a slave unit is the last or the penultimate unit along the product stream, wherein said slave unit is a filtration unit, and wherein the filtration unit is a virus filtration unit.

5. The production plant according to claim 1, wherein at least one of the at least two slave units is a residence time components unit.

6. The production plant according to claim 1, wherein at least one of the at least two slave units is a conditioning component unit.

7. The production plant according to claim 6, wherein the conditioning component unit comprises a conditioning loop.

8. The production plant according to claim 7, wherein the conditioning loop is connected to one of the one or more buffer volumes.

9. The production plant according to claim 1, wherein the one or more buffer volumes, the one or more sensors, the one or more controllers and the one more pumps acting together on the state variable buffer volume are in the same slave unit.

10. The production plant according to claim 1, wherein the production plant comprises flexible pipes in which a liquid flow is conveyed, which is measured through the use of a compensating flow rate measurement.

11. The production plant according to claim 1, wherein the state variable comprises fill level or pressure.
